(19) United States
(12) Patent Application Publication
(10) Pub. No.: US 2020/0178766 A1
(43) Pub. Date: Jun. 11, 2020

(54) ENDOSCOPE

(71) Applicant: OLYMPUS CORPORATION, Tokyo (JP)

(72) Inventor: Hirokazu ICHIHARA, Tokyo (JP)

(73) Assignee: OLYMPUS CORPORATION, Tokyo (JP)

(21) Appl. No.: 16/702,641

(22) Filed: Dec. 4, 2019

Related U.S. Application Data
(63) Continuation of application No. PCT/JP2018/008600, filed on Mar. 6, 2018.

(30) Foreign Application Priority Data
Jun. 8, 2017 (JP) 2017-113590

(51) Int. Cl. A61B 1/00

(52) U.S. Cl. CPC A61B 1/00096; A61B 1/00193; A61B 1/00009

(57) ABSTRACT

An endoscope includes: an observation through hole which is formed in a distal end portion body, an opening shape of the observation through hole being defined by a pair of long sides which face each other and a pair of short sides which face each other; grooves for cutting machining clearance, the grooves being formed at four corners where the long sides and the short sides respectively intersect with each other during machining the observation through hole; an observation lens being inserted into the observation through hole; and an adhesive material which is circumferentially disposed between the observation through hole and the observation lens. In such an endoscope, each groove is formed in contact with the short sides, and projects toward the outside of the observation through hole from the long sides.

### CROSS REFERENCE TO RELATED APPLICATION

This application is a continuation application of PCT/JP2018/008600 filed on Mar. 6, 2018 and claims benefit of Japanese Application No. 2017-113590 filed in Japan on Jun. 8, 2017, the entire contents of which are incorporated herein by this reference.

### BACKGROUND OF INVENTION

### 1. Field of the Invention

The present invention relates to an endoscope having a plurality of optical systems at a distal end portion of the endoscope.

### 2. Description of the Related Art

Recently, in fields such as a medical endoscope and an industrial endoscope, needs for stereoscopic observation of a subject using a stereoscopic image pickup unit have been increasing.

To satisfy such needs, there has been proposed an endoscope where a stereoscopic image pickup unit is incorporated in a distal end portion of the endoscope. The stereoscopic image pickup unit used in the endoscope is, in general, formed by arranging a pair of image pickup units in a lateral direction, wherein each image pickup unit includes an optical lens system formed of a lens group including an object lens, a solid-state image pickup device, and a mounting board on which various circuit parts are mounted.

In such an endoscope having the stereoscopic image pickup unit, it is necessary to arrange a pair of image pickup apparatuses side by side. Accordingly, there is a tendency that a diameter of a distal end portion of the endoscope is increased. To suppress the increase of the diameter of the distal end portion, it is necessary to set a distance between optical axes of two image pickup units short. However, when the distance between the optical axes is decreased, it is difficult to ensure a space for liquid-hermetically bonding to a housing circumferences of optical members positioned at most distal ends of the respective optical systems.

On the other hand, for example, as disclosed in Japanese Patent Application Laid-Open Publication No. 2000-10023, it may be possible to adopt a configuration where optical members (front-end lenses) positioned at most distal ends of a pair of optical systems are integrally formed using an optical member having a rectangular shape as viewed in a plan view.

In forming a holding hole which corresponds to such an optical member having a rectangular shape by milling or the like, four corners of the holding hole are formed in a circular arc shape corresponding to an outer diameter of a milling cutter (that is, four corners of the holding hole cannot be worked to have a size equal to or less than the outer diameter of the milling cutter and hence, a so-called “rounded corner” is formed at four corner portions of the holding hole). Accordingly, in order to enable accommodation of the optical member having a rectangular shape in the holding hole by eliminating such rounded corners, a groove for machining clearance which projects in a diagonal direction is formed, in general, on each corner of the above-mentioned holding hole.

### SUMMARY OF THE INVENTION

According to an aspect of the present invention, there is provided an endoscope which includes: a housing, the housing including a distal end surface formed on a distal end portion of an insertion portion, and a holding hole formed in the distal end surface, an opening shape of the holding hole being defined by a pair of long sides which face each other and a pair of short sides which face each other; grooves for cutting machining clearance, the grooves being formed at four corners where the long sides and the short sides respectively intersect with each other during cutting machining of the holding hole; an optical member having a plan view shape similar to the opening shape of the holding hole, the optical member being inserted into the holding hole; and an adhesive material filled between an inner peripheral surface of the holding hole and an outer peripheral surface of the optical member and in the groove portions, wherein each of the grooves is formed in contact with the short sides, and projects toward an outside of the holding hole from the long sides so as to transmit a shrinkage load of the adhesive material filled in the grooves to only a surface of the optical member on a long-side side.

### BRIEF DESCRIPTION OF THE DRAWINGS

FIG. 1 is a perspective view showing the overall configuration of an endoscope system;

FIG. 2 is an end face view of a distal end portion of an endoscope;

FIG. 3 is a cross-sectional view of the distal end portion of the endoscope taken along a line in FIG. 2;

FIG. 4 is an exploded perspective view of a stereoscopic image pickup unit and an illumination unit held on the distal end portion of the endoscope;

FIG. 5 is an explanatory diagram of an observation through hole worked by a milling cutter;

FIG. 6 is an explanatory diagram of a stress generated at a VI portion in FIG. 3;

FIG. 7 is an explanatory diagram showing a cross-sectional area of an observation lens which receives a stress on a long-side side of the observation lens;

FIG. 8 is an explanatory diagram of an observation through hole worked by a milling cutter according to a first modification of the endoscope;

FIG. 9 is an end face view of a distal end portion of an endoscope according to a second modification of the endoscope; and

FIG. 10 is an end face view of a distal end portion of an endoscope according to a third modification of the endoscope.

### DETAILED DESCRIPTION OF THE PREFERRED EMBODIMENT

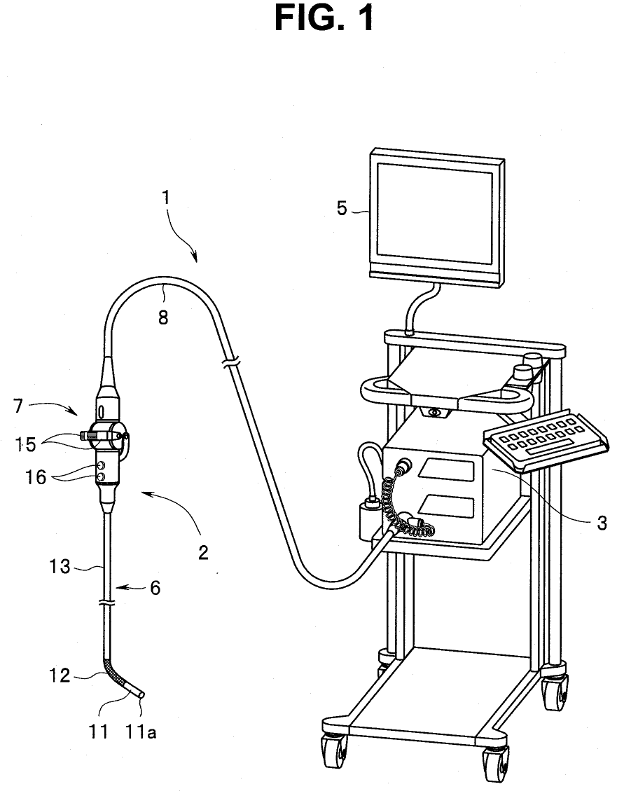

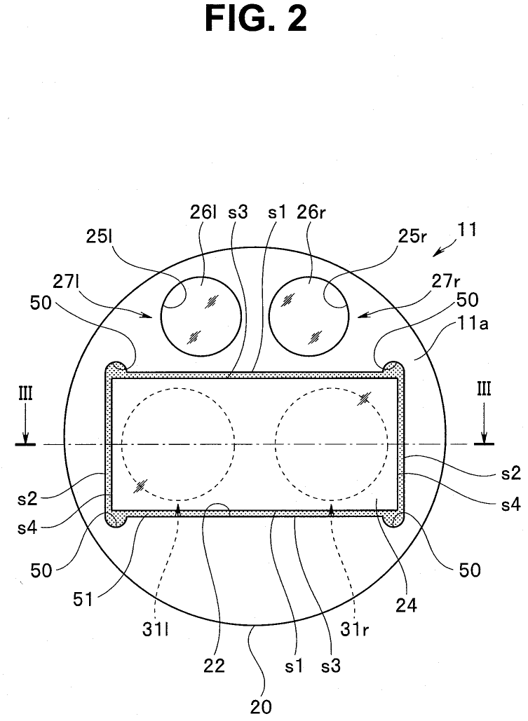

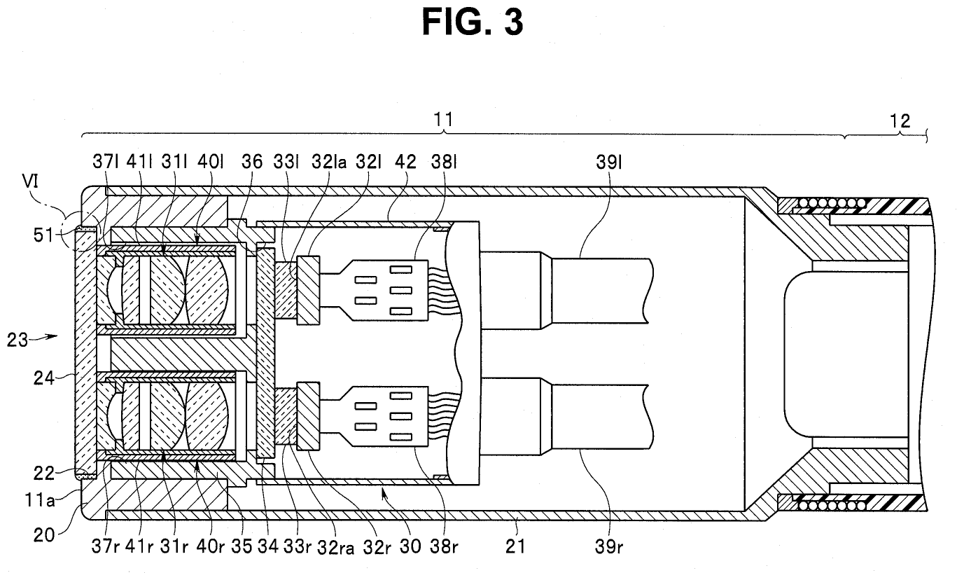

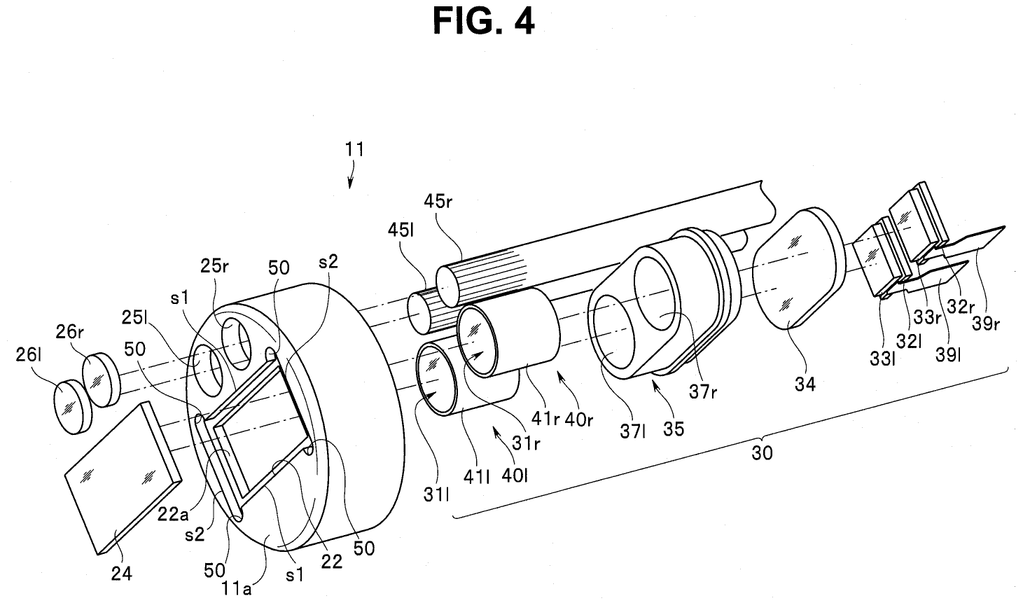

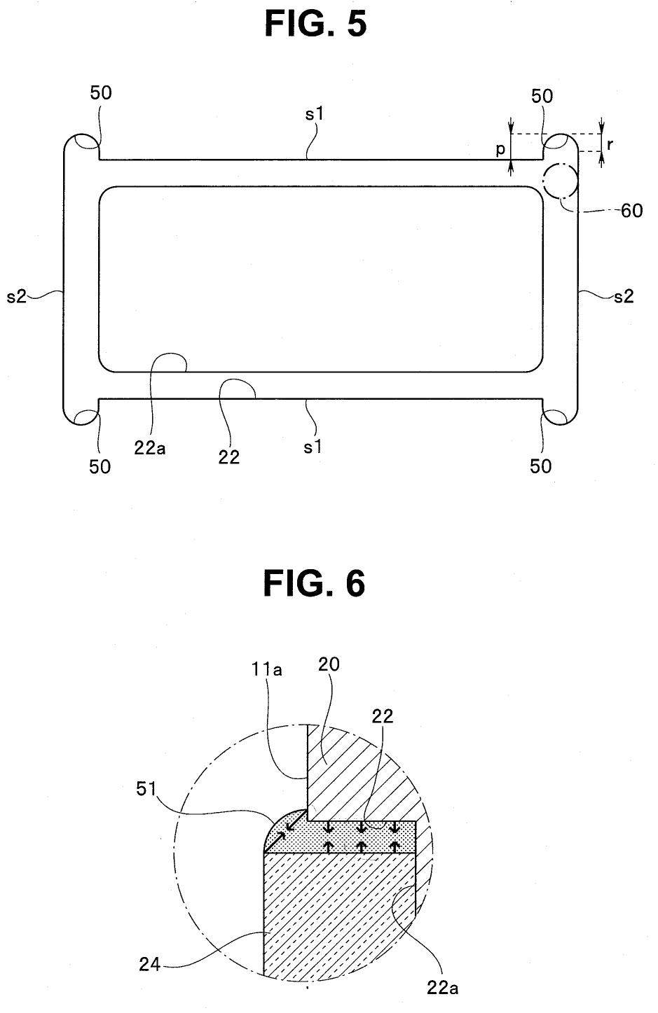

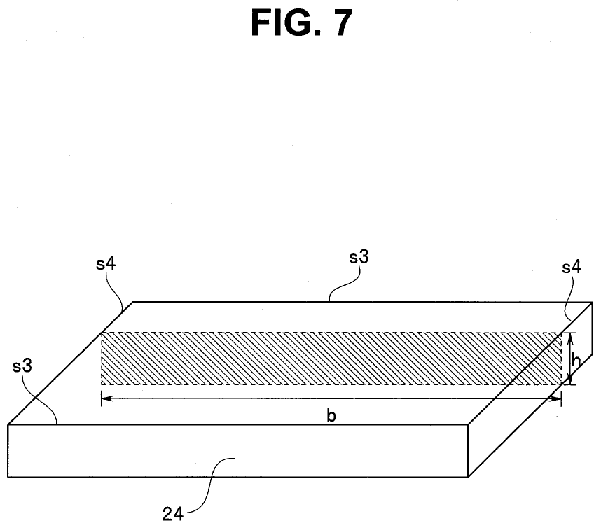

Hereinafter, a mode of the present invention is described with reference to drawings. The drawings relate to one embodiment of the present invention. FIG. 1 is a perspective view showing the overall configuration of an endoscope system. FIG. 2 is an end face view of a distal end portion of an endoscope. FIG. 3 is a cross-sectional view of the distal end portion of the endoscope taken along a line in FIG. 2. FIG. 4 is an exploded perspective view of a stereoscopic image pickup unit and an illumination unit held on the distal end portion of the endoscope. FIG. 5 is an explanatory diagram of an observation through hole worked by a milling cutter. FIG. 6 is an explanatory diagram of a stress generated at a VI portion in FIG. 3. FIG. 7 is an explanatory diagram showing a cross-sectional area of an observation lens which receives a stress on a long-side side.

The endoscope system 1 shown in FIG. 1 includes: an endoscope 2 (stereoscopic endoscope) which is capable of stereoscopically picking up an image of a subject from different viewpoints; a processor 3 to which the endoscope 2 is detachably connected; and a monitor 5 which forms a display device and displays an image signal formed by the processor 3 as an endoscope image.

The endoscope 2 according to this embodiment is, for example, a rigid endoscope used in a laparoscopic surgery. The endoscope 2 includes an elongated insertion portion 6, an operation portion 7 continuously connected to a proximal end side of the insertion portion 6, a universal cable 8 extending from the operation portion 7 and connected to the processor 3.

In forming the insertion portion 6, a distal end portion 11 formed of mainly a metal member made of stainless steel or the like, a bending portion 12, and a rigid tube portion 13 formed of a metal tube made of stainless steel or the like are continuously connected in order from a distal end side of the insertion portion 6.

The insertion portion 6 is a portion which is inserted into a body of the subject, and a stereoscopic image pickup unit 30 (see FIG. 3 and the like) for stereoscopically picking up an image of the inside of the subject is incorporated in the distal end portion 11. In the bending portion 12 and the rigid tube portion 13, image pickup cable bundles 391, 39r (see FIG. 3) electrically connected to the stereoscopic image pickup unit 30, a light guide bundles 451, 45r (see FIG. 4) for transmitting an illumination light to the distal end portion 11 and the like are inserted. As the endoscope 2 according to this embodiment, a rigid endoscope in which a portion of the endoscope which is disposed on a more proximal end side than the bending portion 12 is formed of the rigid tube portion 13 is described as an example. However, the endoscope 2 is not limited to such a configuration, but the endoscope 2 may be a soft endoscope where the portion of the endoscope disposed on a more proximal end side than the bending portion 12 is formed of a flexible tube portion having flexibility.

An angle lever 15 which performs a remote control of the bending portion 12 is mounted on the operation portion 7, and various switches 16 for operating a light source device of the processor 3, a video system center and the like are mounted on the operation portion 7.

The angle lever 15 is bending operation means capable of bending the bending portion 12 of the insertion portion 6 in four directions, that is, upward, downward, leftward, and rightward directions in this embodiment. Note that the bending portion 12 is not limited to the configuration where the bending portion 12 is bendable in four directions, that is, upward, downward, leftward, and rightward directions. For example, the bending portion 12 may have the configuration where the bending portion 12 is bendably operable only in two directions, that is, upward and downward directions, or leftward and rightward directions.

Next, the configuration of the distal end portion 11 of such an endoscope 2 is described in detail with reference to FIG. 2 and FIG. 3.

As shown in FIG. 3, the distal end portion 11 has a distal end portion body 20 which forms a housing having an approximately circular columnar shape and a distal end cylindrical body 21 having an approximately circular cylindrical shape and having a distal end which is fixed to the distal end portion body 20. In such a configuration, the distal end of the distal end cylindrical body 21 is fitted on an outer periphery of the distal end portion body 20, and an end face of the distal end portion body 20 which is exposed from the distal end cylindrical body 21 forms a distal end surface 11a of the distal end portion 11.

As shown in FIG. 2 and FIG. 3, an observation through hole 22 which opens to the distal end surface 11a as a holding hole is provided in the distal end portion body 20. The observation through hole 22 is formed of a through hole, an opening shape of which is defined by a pair of upper and lower long sides s1 which face each other, and a pair of left and right short sides s2 which face each other, for example. In other words, the observation through hole 22 according to the embodiment is formed of the through hole where the opening shape is formed in a laterally elongated rectangular shape (that is, a rectangular shape elongated in the lateral bending direction of the bending portion 12).

An observation lens 24 which forms an optical member is water-tightly fixed to the observation through hole 22. Accordingly, an observation window 23 is formed on the distal end surface 11a of the distal end portion 11. Note that the observation lens 24 in this embodiment is, for example, a lens in a broad sense including a flat glass, a curved glass having a predetermined optical power and the like. In this embodiment, the observation lens 24 is formed of a flat glass having a similar shape to the opening shape of the observation through hole 22, for example. In other words, the observation lens 24 is formed of a flat glass having a plan view shape defined by a pair of upper and lower long sides s3 which face each other and a pair of left and right short sides s4 which face each other and having a similar shape to the observation through hole 22 (a relationship of s1:s3=s2:s4 being established).

On a back surface side of the observation lens 24 (that is, the observation window 23), a distal end side of a pair of object optical systems (first and second object optical systems 311, 31r) which form the stereoscopic image pickup unit 30 can be disposed.

For example, as shown in FIG. 2, above the observation through hole 22 (that is, above the observation through hole 22 in the vertical bending direction of the bending portion 12), illumination through holes 251, 25r which form a pair are formed in the distal end surface 11a of the distal end portion body 20 side by side in the lateral direction. Illumination optical systems 271, 27r which form a pair and are optically connected to the light guide bundles 451, 45r which form a pair are respectively held in the respective left and right illumination through holes 251, 25r. With such a configuration, illumination windows 261, 26r are formed at the distal end surface 11a of the distal end portion 11.

As shown in FIG. 2 and FIG. 4, the stereoscopic image pickup unit 30 includes: the above-mentioned first object optical system 311 and second object optical system 31r which are disposed side by side in the lateral direction in the observation window 23; a first image pickup device 321 which receives an optical image which the first object optical system 311 forms (first optical image); a second image pickup device 32r which receives an optical image which the second object optical system 31r forms (second optical image); a single centering glass 34 which is arranged on optical paths of the first and second optical images, and forms an optical member to which respective light receiving surface 321a, 32ra sides of the first and second image pickup devices 321, 32r are positioned and fixed by adhesion; a holding frame 35 which holds the first and second image pickup devices 321, 32r by way of the centering glass 34.

The first and second image pickup devices 321, 32r are, for example, formed of a solid-state image pickup device such as a charge coupled device (CCD) or a complementary metal oxide semiconductor (CMOS). Cover glasses 331, 33r for protecting the light receiving surfaces 321a, 32ra are fixed by adhesion to the first and second image pickup devices 321, 32r.

Flexible printed circuit boards (FPC boards) 381, 38r are electrically connected to terminal portions (not shown in the drawing) mounted on the first and second image pickup devices 321, 32r respectively. On the respective FPC boards 381, 38r, for example, various electronic parts such as a digital IC for generating a drive signal to the image pickup device, an IC drive power source stabilizing capacitor for stabilizing a drive power source for the digital IC, and resistors are respectively mounted by soldering or the like. The image pickup cable bundles 391, 39r are electrically connected to the respective FPC boards 381, 38r.

Note that in this embodiment, the first and second image pickup devices 321, 32r, the respective FPC boards 381, 38r on which various electronic parts are mounted, and distal end sides of the respective image pickup cable bundles 391, 39r which are electrically connected to the respective FPC boards 381, 38 are integrally covered by a single cover body 42.

The centering glass 34 is formed of a transparent glass substrate extending in the lateral direction of the distal end portion 11. The light receiving surface 321a, 32ra sides of the first and second image pickup devices 321, 32r are respectively fixed to the centering glass 34 by way of the cover glasses 331, 33r.

More specifically, the first and second image pickup devices 321, 32r are positioned and fixed to the centering glass 34 by bonding the cover glasses 331, 33r laminated to the light receiving surfaces 321a, 32ra to the centering glass 34 by way of a ultraviolet curing transparent adhesive agent (UV adhesive agent) or the like in a state where the first and second image pickup devices 321, 32r are spaced apart from each other by a predetermined distance therebetween. Further, a distal end side of the cover body 42 is fixedly mounted on a glass holding portion 36.

The holding frame 35 is formed of, for example, a columnar metal member having an approximately rectangular shape with rounded corners in a cross-sectional shape (for example, see FIG. 4). The glass holding portion 36 is recessed on a proximal end side of the holding frame 35, and the centering glass 34 is fixed to the glass holding portion 36 by an adhesive agent or the like.

For example, as shown in FIG. 3 and FIG. 4, a first object optical system holding hole 371 and a second object optical system holding hole 37r are formed in the holding frame 35 in a state where the first object optical system holding hole 371 and the second object optical system holding hole 37r are arranged side by side in the lateral direction with a preliminarily set distance therebetween. These first and second object optical system holding holes 371, 37r are formed of through holes where the distal end sides of the first and second object optical system holding holes 371, 37r open at an end face (distal end surface 11a) of the holding frame 35 and proximal end sides of the first and second object optical system holding holes 371, 37r communicate with the glass holding portion 36.

The first and second object optical systems 311, 31r are held by the first and second object optical system holding holes 371, 37r respectively in a state where the first and second object optical systems 311, 31r are formed into units as first and second object optical system units 401, 40r.

In other words, the first and second object optical systems 311, 31r form the first and second object optical system units 401, 40r in a state where the first and second object optical systems 311, 31r are held by first and second lens frames 411, 41r. By positioning and fixing the first and second object optical system units 401, 40r in the first and second object optical system holding holes 371, 37r by way of an adhesive agent or the like, the first and second object optical systems 311, 31r are integrally held together with the first and second image pickup devices 321, 32r by the single holding frame 35.

Next, the configuration of the observation window 23 which integrally covers the first and second object optical systems 311, 31r at the distal end portion 11 provided with the above-mentioned stereoscopic image pickup unit 30 is described in more detail.

In the observation window according to this embodiment, the observation through hole 22 which holds the observation lens 24 is formed, for example, by cutting the distal end portion body 20 using a tool such as a milling cutter 60.

In this case, the observation through hole 22 according to this embodiment has a rectangular opening shape. To prevent the formation of a so-called rounded corner having a circular arc shape at four corners where respective long sides s1 and the respective short sides s2 intersect with each other, grooves 50 for machining clearance are formed. In other words, the grooves 50 for machining clearance are formed at four corners of the observation through hole 22 for eliminating the rounded corners which obstruct the insertion of the observation lens 24.

Each of these grooves 50 is formed in contact with the short side s2, and projects toward the outside of the observation through hole 22 from the long side s1. More specifically, each groove 50 is formed so as to project toward a tangential direction of the short side s2 (an extending direction of the short side s2) at an imaginary intersecting point between the long side s1 and the short side s2. In such a configuration, as shown in FIG. 5, in order to eliminate the rounded corner with certainty, it is desirable that a projecting amount p of each groove 50 from the long side s1 be set larger than a radius r of the milling cutter 60.

In machining the observation through hole 22 according to this embodiment, an inwardly directed flange 22a which positions the observation lens 24 in an optical axis direction is integrally formed with an inner periphery of the observation through hole 22. Even when rounded corners are formed on the inwardly directed flange 22a, such rounded corners do not particularly cause a problem with respect to insertion property or the like of the observation lens 24. Accordingly, a groove for machining clearance is not formed at four corners of the inwardly directed flange 22a.

As shown in FIG. 2 and FIG. 3, the observation lens 24 is inserted into the observation through hole 22 formed in this manner, the observation lens 24 is bonded to the observation through hole 22 by way of an adhesive portion 51 such as soldering which is formed circumferentially between an inner peripheral surface of the observation through hole 22 and an outer peripheral surface of the observation lens 24. Note that an adhesive agent can be used in place of solder as the adhesive portion 51.

In the observation window 23 having such a configuration, the adhesive material 51 formed of solder or the like is shrunken during curing. Accordingly, for example, as shown in FIG. 6, a stress having a component in a vertical direction and a component in a bending direction acts on a side surface of the observation lens 24 due to a shrinkage load generated in the adhesive material 51.

In this case, a volume of the adhesive material 51 in the groove 50 is large compared to volumes of other portions and hence, a larger shrinkage load is generated in the groove 50 than other portions. However, the groove 50 according to this embodiment is formed such that the groove 50 projects from a long side s1 side of the observation through hole 22 and hence, a large load generated in the groove 50 is received by a surface on a long side s3 side having a large cross-sectional area whereby it is possible to prevent an excessively large stress from acting on a short side s4 side of the observation lens 24.

In other words, when a predetermined shrinkage load acts on one side surface of the observation lens 24, a normal stress inversely proportional to a cross-sectional area acts on the side surface and hence, a normal stress per unit area becomes smaller on the long side s3 side than the short side s4 side.

More specifically, for example, as shown in FIG. 7, a length of a side of the observation lens 24 on the long side s3 side is larger than a length of a side of the observation lens 24 on the short side s4 side. Accordingly, a cross-sectional area (bxh) on the long side s3 side with the length of the side set as b and a height of the side set as h is larger than a corresponding cross-sectional area on the short side s4 side. Since it is known that a normal stress per unit area is inversely proportional to a cross-sectional area (normal stress=load÷cross-sectional area), a normal stress per unit area can be made smaller on the long side s3 side than the short side s4 side even when a similar shrinkage load is generated.

When a bending moment based on a predetermined shrinkage load acts on one side surface of the observation lens 24, a bending stress inversely proportional to a section modulus acts on the side surface and hence, a bending stress per unit area becomes smaller on the long side s3 side than the short side s4 side.

More specifically, the length of the side of the observation lens 24 on the long side s3 side is larger than the length of the side of the observation lens 24 on the short side s4 side. Accordingly, a section modulus (b×h²÷6) on the long side s3 side with the length of the side set as b and the height of the side set as h is larger than a corresponding section modulus on the short side s4 side. Since it is known that a bending stress per unit area is inversely proportional to a section modulus (bending stress=bending moment÷section modulus), a bending stress per unit area can be made smaller on the long side s3 side than the short side s4 side even when a similar bending moment is generated.

According to this embodiment, the endoscope 2 includes: the observation through hole 22 which is formed in the distal end portion body 20 having the distal end surface 11a formed on the distal end portion 11 of the insertion portion 6, the observation through hole 22, an opening shape of which is defined by the pair of long sides s1 which face each other and the pair of short sides s2 which face each other; the grooves 50 for machining clearance which are formed at four corners where the long sides s1 and the short sides s2 respectively intersect with each other during machining the observation through hole 22; the observation lens 24 which has a plan view shape similar to the opening shape of the observation through hole 22 and is inserted into the observation through hole 22; and the adhesive material 51 which is circumferentially disposed between the inner peripheral surface of the observation through hole 22 and the outer peripheral surface of the observation lens 24. In such an endoscope 2, by forming each groove 50 in contact with the short side s2 and projecting toward the outside of the observation through hole 22 from the long side s1, in fixing the observation lens 24 to the observation through hole 22 having the grooves 50 for machining clearance at the intersecting portions of the respective sides in two pairs having different lengths, it is possible to prevent breaking of the observation lens 24 caused by shrinking of the adhesive material 51.

In other words, each groove 50 for machining clearance is projected from the long side s1 side and hence, a shrinkage load of the adhesive material 51 filled in the groove 50 is received only by the surface on the long side s3 side having a large cross-sectional area (and a section modulus) in the observation lens 24. Accordingly, it is possible to prevent a large stress from acting on the short side s4 side of the observation lens 24 and hence, breaking of the observation lens 24 can be prevented.

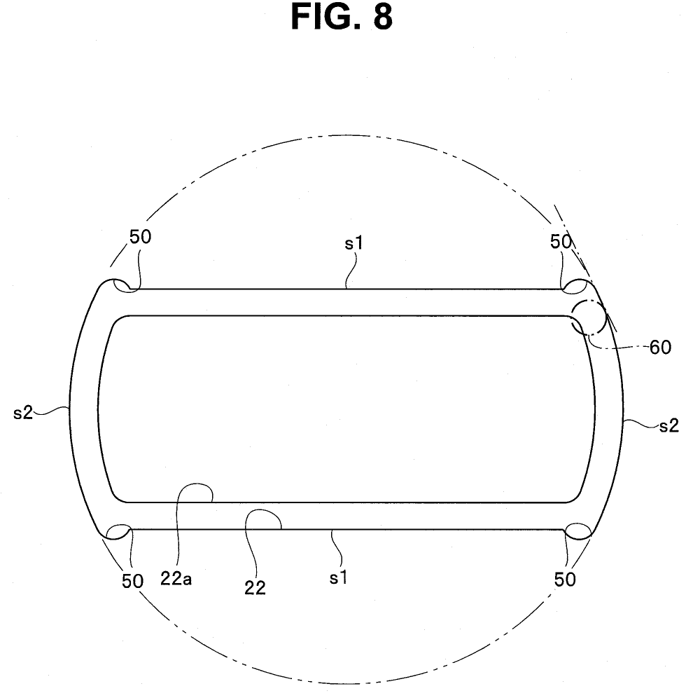

In this embodiment, for example, as shown in FIG. 8, a pair of short sides s2 of an observation through hole 22 (and a pair of short sides s4 of an observation lens 24) may be formed into a circular arc shape. In this case, each groove 50 is also formed in contact with the short side s2 and projects toward the outside of the observation through hole 22 from a long side s1. More specifically, each groove 50 is formed so as to project in a tangential direction of the short side s2 (an extending direction of the short side s2) at an imaginary intersecting point between the long side s1 and the short side s2. Note that, in such a configuration, it is desirable that the respective short sides s2 (and the respective short sides s4) be circular arcs disposed on a concentric circle.

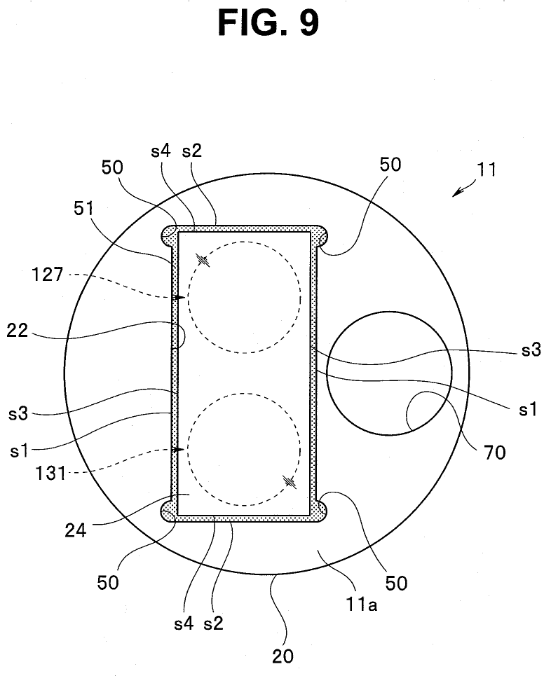

In the above-mentioned embodiment, the present invention has been described by taking the configuration where the present invention is applied to a stereoscopic endoscope as an example. However, for example, as shown in FIG. 9, the present invention is also applicable to an endoscope provided with a monocular observation optical system 131 for normal observation. In this case, an observation window 23 is arranged so as to be elongated in a vertical direction, and for example, the observation optical system 131 and an illumination optical system 127 can be arranged on a back surface side of the observation window 23. Note that, in the endoscope shown in FIG. 9, a channel opening portion 70 which communicates with a treatment instrument channel not shown in the drawing is formed on a distal end surface 11a on a side of the observation optical system 131 and the illumination optical system 127.

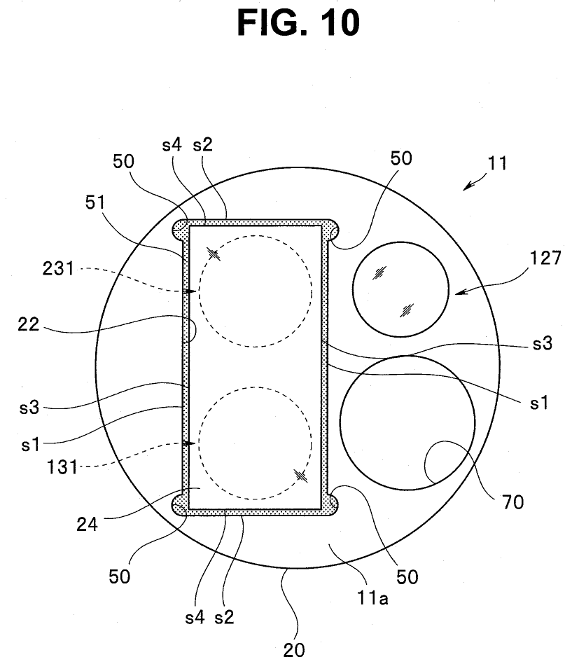

Further, for example, as shown in FIG. 10, the present invention is also applicable to an endoscope provided with a monocular observation optical system 131 for normal observation and a monocular observation optical system 231 for particular observation. In this case, an observation window 23 is arranged so as to be elongated in a vertical direction, and for example, the observation optical systems 131, 231 can be arranged on a back surface side of the observation window 23. Note that, in the endoscope shown in FIG. 10, an illumination optical system 127 and a channel opening portion 70 are formed on a distal end surface 11a on a side of the observation optical systems 131, 231.

Note that the present invention is not limited to the above-mentioned respective embodiments, and various modifications and changes are conceivable within the technical scope of the present invention.

What is claimed is:

1. An endoscope comprising:
a housing, the housing including a distal end surface formed on a distal end portion of an insertion portion, and a holding hole formed in the distal end surface, an opening shape of the holding hole being defined by a pair of long sides which face each other and a pair of short sides which face each other;
grooves for cutting machining clearance, the grooves being formed at four corners where the pair of long sides and the pair of short sides respectively intersect with each other during cutting machining of the holding hole;
an optical member having a plan view shape similar to the opening shape of the holding hole, the optical member being inserted into the holding hole; and
an adhesive material filled between an inner peripheral surface of the holding hole and an outer peripheral surface of the optical member and in the groove portions, wherein
each of the grooves is formed in contact with the short sides, and projects toward an outside of the holding hole from the long sides so as to transmit a shrinkage load of the adhesive material filled in the grooves to only a surface of the optical member on a long-side side.

2. The endoscope according to claim 1, wherein the optical member integrally covers distal ends of two optical systems held in the housing.

3. The endoscope according to claim 1, wherein both the pair of long sides and the pair of short sides are straight lines.

4. The endoscope according to claim 1, wherein the pair of long sides are straight lines, and the pair of short sides are circular arcs disposed on a concentric circle.

5. The endoscope according to claim 1, wherein a projecting amount of each of the grooves from each of the pair of long sides is larger than a radius of a tool for forming the holding hole by cutting machining.

6. The endoscope according to claim 1, wherein a projecting direction of each of the grooves is a tangential direction of each of the pair of short sides.
